(19) United States
(12) Patent Application Publication
(10) Pub. No.: US 2014/0187954 A1
(43) Pub. Date: Jul. 3, 2014

(54) INFANT BONE ASSESSMENT

(71) Applicant: Mayo Foundation for Medical Education and Research, Rochester, MN (US)

(72) Inventors: Mostafa Fatemi, Rochester, MN (US); Armen Sarvazyan, West Trenton, NJ (US)

(21) Appl. No.: 14/141,747

(22) Filed: Dec. 27, 2013

Related U.S. Application Data
(60) Provisional application No. 61/748,194, filed on Jan. 2, 2013.

(51) Int. Cl. A61B 5/00; A61B 8/08

(52) U.S. Cl. CPC A61B 5/4504; A61B 8/48
USPC 600/449

(57) ABSTRACT

System and method for assessment of demineralization of a bone of a subject. The acoustic wave, propagating along the bone as a result of generation, at the bone, of the ultrasound radiation force by a transducer of the ultrasound system, is outcoupled at a predetermined location along the bone and detected with an acoustic receiver. Time-dependent frequency and temporal characteristics of so detected wave contain data representing a bone demineralization characteristic.

### CROSS-REFERENCE TO RELATED APPLICATIONS

The present application claims priority from and benefit of U.S. Provisional Patent Application No. 61/748,194 filed on Jan. 02, 2013 and titled “Infant Bone Assessment”. The disclosure of the above-identified patent application is incorporated herein by reference in its entirety.

### BACKGROUND OF THE INVENTION

The present invention relates to use of non-invasive methodologies and, in particular, of ultrasound system and method for assessment of the condition of infant bone.

### SUMMARY OF THE INVENTION

Embodiments of the invention provide an ultrasound system for assessment of a structural parameter of a bone of a subject, the system including a signal generator and a source of ultrasound wave. The source of ultrasound wave is driven by the signal generator and oriented repositionably and distantly with respect to the subject to apply an ultrasound radiation force to an identified input location at the bone to create an acoustic wave propagating along the bone. Optionally, the source is structured to transmit, to the bone, a substantially focused ultrasound beam. The system further includes an acoustic detector positioned moveably along the bone and enabled to detect the acoustic wave at a first and second output positions along the bone. The acoustic detector may be operably adapted for detection of the acoustic wave while placed in contact with the skin of the subject.

The system additionally contains electronic circuitry processor configured to record spectral and temporal data representing said acoustic waves based at least on the output from the acoustic detector and to process said spectral and temporal data to determine a structural parameter of the bone. A specific example of such data-processing electronic circuitry includes a processor programmed with a specific program code effectuating such calculation of a structural parameter of the bone. In one case, the structural parameter represents a change in mineralization characteristic of said bone. In a related implementation, the signal generator is adapted to drive the source of ultrasound in at least one of a short pulse mode; a tone-burst mode; and an amplitude-modulated tone-burst mode. Alternatively or in addition, the spectral and temporal data recorded with the use of the data-processing electronic circuitry include at least one of a mean change in frequency as a function of time; a parameter representing a broadening of a frequency spectrum as a function of time; a mean time of arrival of said acoustic wave from the identified input location to a position along the bone at which said acoustic wave is detected; a mean time of arrival of said acoustic wave, calculated for each individual frequency component, from the identified input location to a position along the bone at which said acoustic wave is detected; a parameter representing a change in a temporal characteristic of said acoustic wave as a function of frequency (for example the time duration of the wave for each frequency component); and a change in the amplitude of said acoustic wave determined in a time-frequency domain.

### BRIEF DESCRIPTION OF THE DRAWINGS

FIG. 1 is a diagram illustrating a conventional QUS system.

FIG. 2 is a diagram illustrating formation of ultrasound radiation force at the bone and measurement of acoustic characteristics of the bone according to an embodiment of the invention.

FIGS. 3A and 3B are diagrams schematically representing related embodiments of measurements of acoustic characteristics of a bone.

FIGS. 4A, 4B, and 4C are spectrograms representing an acoustic wave that has propagated through the same bone sample (which is subject to demineralization) and acquired with the embodiment of FIG. 3A. FIG. 4A: intact bone sample. FIG. 4B: bone sample demineralized for 4 days. FIG. 4C: bone sample demineralized for 7 days.

FIGS. 5A, 5B, and 5C are spectrograms representing an acoustic wave that has propagated through the same bone sample (which is subject to demineralization) and acquired with the embodiment of FIG. 3B. FIG. 5A: intact bone sample. FIG. 5B: bone sample demineralized for 4 days. FIG. 5C: bone sample demineralized for 7 days.

FIG. 6 is a plot illustrating a change in weighted mean frequency of an acoustic wave (detected, upon propagation from the point of application of the URF to the bone to the output end of the bone, with a hydrophone) as a function of time associated with bone demineralization process.

FIG. 7 is a diagram illustrating the geometry of the propagation of the acoustic wave (formed with the URF according to an embodiment of the invention) through the bone and time-dependences of acoustic signals acquired with a hydrophone bone at different output locations.

FIG. 8 is a diagram presenting an alternative embodiment of an ultrasound system for bone evaluation.

FIG. 9 is a diagram illustrating schematically an example of an ultrasound sub-system for use with an embodiment of the invention.

FIG. 10 is a flow-chart representing an embodiment of a method of the invention.

### DETAILED DESCRIPTION

Embodiments of the present invention are directed to a new type of quantitative ultrasound technique that is operationally compatible with an infant subject, does not cause significant stress in gentle infant skin, and allows the evaluation of infant bone properties and an early detection of bone pathology.

Bones naturally become thinner as people grow older (and, beginning in the middle age, existing bone cells are known to be reabsorbed by the body faster than new bone material is generated). As this occurs, the bones lose minerals and with them mass and structure, which makes the bones weaker and increases the risk of bone breaking.

The term “osteopenia” refers to bone mineral density (BMD) that is lower than normal peak BMD but not low enough to be classified as osteoporosis, to which osteopenia is considered to be a precursor. More specifically, osteopenia is defined as a bone mineral density T-score between about −1.0 and about −2.5.

Quantitative characterization of bone condition in pediatrics is of importance, particular in neonatology, where the use of conventional densitometry methods on infants and newborns is limited. Significance of assessing skeletal systems of newborns has become particularly pronounced with growing emphasis on osteopenia of prematurity (that is, the decrease of bone mass and density in premature and low-birth-weight infants. According to some studies, deviations in bone metabolism and development are observed in up to a half of low-birth-weight and premature newborns, which amounts to up to 5 out of every 100 newborns. One of the pathogenic factors contributing to osteopenia of prematurity, the onset of which is between about 6 and 12 weeks postnatal, remains an insufficient mineral intake of calcium and phosphorous.

Regular screening, which helps to identify infants developing neonatal rickets, enables neopatologists to minimize risk factors and optimize nutrition and mineral supplementation of the subjects. While a variety of ways exists for subject screening, an optimal methodology has not been identified yet. For example, biochemical measurements (associated with serum calcium, inorganic phosphorus, and alkaline phosphatase or ALP, for instance) correlates poorly with bone mineralization. While osteopenia of prematurity (OP) is hallmarked with radiographic evidence of decreased bone mineralization, the X-ray methodologies have a drawback of low sensitivity. Studies indicates that the decrease of bone mineralization has to reach a level of about 20% to 40% before it can be reliably detected with conventional X-ray approach, and that the success of such detection varies from about 28 to about 45 percent, depending on the type of X-ray equipment machine. The practical, clinical utility of the use of standard X-ray modality is disputed and argued to be not particularly useful in diagnosing osteopenia, because the standard X-ray modality is not sensitive enough to detect small amounts of bone loss or minor changes in bone density. Scans carried out with dual energy X-ray absorptiometry (or DEXA), which is commonly recognized as a method of choice for bone mass measurements in adults, are more sensitive in detecting small changes in bone mass density (BMD) and, while its use with pre-term and term infants has been validated, the radiation exposure of the subject remains a serious drawback.

Conventional radiography and quantitative ultrasonography (QUS) are additional methods that could be used for the purposes of OP screening. Due to the use of ionizing radiation and bulky equipment, however, the radiography is not suitable for infants and especially for the low-birth-weight pre-term neopates (which is a group with a particular risk of mineral compromise in bones). The QUS is designed to measure the speed of sound in large extremity bones by transmitting a single-frequency ultrasound tone wave (for example, at about 1.25 MHz) to one end of the bone and measuring the arrival time interval at the other end of the bone and, therefore, is adapted for adult bone assessment and is not readily reconfigurable for use with infants. In addition, the accuracy of the QUS results are known to be affected by the presence of subcutaneous tissues and, in particular, by the subcutaneous fat layer. Moreover, the current QUS technology employs and is limited by a single-frequency mode of operation. Furthermore, when the structure of the bone is measured in a low-frequency range, large transducers are required the use of which is impractical for infant bone assessment.

It may be concluded, therefore, that at the present time there exists no screening test that has been shown to both be sensitive and provide specific evidence of OP development over the first several weeks of life of a premature infant. There remains a need in developing advanced QUS methodologies free of the known operational artifacts and applicable to assessment of infant bone structure.

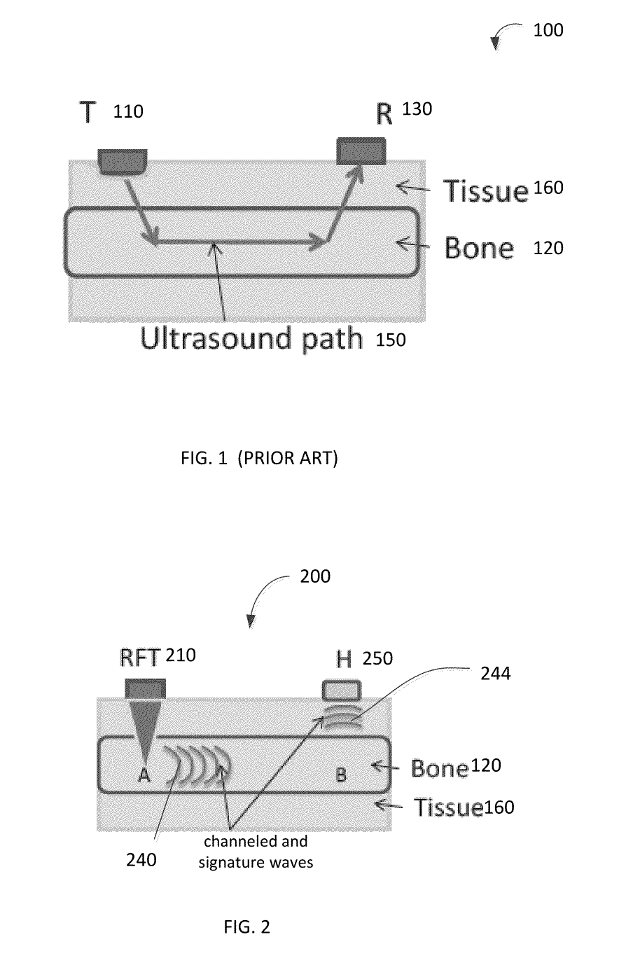

In reference to FIG. 1, a conventional axial QUS technique typically employs two substantially large transducers: one (T, 110) to transmit ultrasound to the bone 120 and another (R, 130) to receive the ultrasound transmitted along the path 150, with the purpose of determining the speed of sound in the bone 120. With such approach to the measurement, the ultrasound wave has to pass through the subcutaneous soft tissue 160 before and after is passes through the bone 120. Because the soft tissue varies from patient to patient, the passage of the ultrasound through the tissue 160 causes errors in the bone speed-of-sound measurements. In addition, it is recognized that in order to reliably transmit the ultrasound into the bone, the transducer 110 has to be firmly pressed to the skin of the patient, which is unacceptable in the case of the infant's delicate skin. The wave propagation in the bone material is highly dispersive, and such propagation is strongly affected by the geometry and material content of the bone (especially at low acoustic frequencies). Accordingly, the information acquired with the use of a traditional single-frequency approach 100 is rather limited in terms of its representation of the bone properties.

In contradistinction with the conventional QUS 100, schematically shown in FIG. 1, an embodiment 200 of FIG. 2 utilizes the ultrasound radiation force, exerted by ultrasound on an object, to excite the propagation of a wave through the bone. According to the idea of the invention, the nonlinear acoustic methodology is used, which stems from employing a nonlinear phenomenon in acoustic wave propagation to remotely exert a localized, inside the subject, vibrating force at a pre-determined frequency. The nonlinear mechanism of such so-called ultrasound radiation force (or, URF), which may also be referred to as an acoustic radiation force (or ARF) allows for frequency conversion from high to low frequencies without the need or use of large-sized transducers. An application of the URF enables the user to explore the bone response at frequencies much lower than the primary ultrasound frequencies. For example, by projecting a 1-msec tone-burst of focused ultrasound at 3 or 6 MHz one can produce vibrations in the bone at a wide frequency range in the order of tens of kHz. The presently proposed methodology does not rely—in contradistinction with the conventional QUS—on the determination of speed-of-sound. Rather, it uses a combination of time and wide spectral band frequency signatures of acoustic waves propagating through the bone in response to the applied URF. Advantages of the proposed methodology over the QUS include: capability of providing diagnostic information in a wide frequency range, remote and direct delivery of mechanical vibrations to the bone, and a compact ultrasound system implementation, all of which makes the proposed methodology to be uniquely qualified for use with newborns and infants.

The URF is a physical phenomenon associated with the propagation of acoustic waves in a deformable medium; in biomedical applications, the URF is often attributed to the presence of attenuation in the medium (which attenuation includes both scattering and absorption of the ultrasound wave; in soft tissues it is dominated by absorption). In essence, the URF originates from the nonlinear terms in the balance of linear momentum governing the propagation of acoustic waves, resulting in the mean motion of the medium under prescribed (zero-mean) ultrasound excitation. In other words, a transfer of momentum occurs in the direction of wave propagation, which generates a force causing displacement of the tissue (on a time-scale slower than that of the ultrasound wave propagation). For example, a high-intensity ultrasound beam applied to the tissue produces substantially constant average URF. The magnitude, location, spatial extent, and duration of ultrasound radiation force can be controlled to interrogate the mechanical properties of the tissue. The URF is being utilized in medical ultrasonic imaging to generate images based on the mechanical properties of the tissue.

As shown in FIG. 2, a transducer (RFT, 210) is activated to generate an ultrasound wave at a frequency f₀ that creates, at a point A of the bone 120, an URF applied to the bone 120. The applied URF can be generated in a form of transient force (impulse force, for example), harmonic force (such as a sinusoidal force) and at a frequency that may be much lower than f₀. The applied URF produces a wave 240 that travels directly through the bone material. The low-frequency vibration 244 of the bone 120, caused by the propagation of the wave 240, is detected in the vicinity of point B at the bone 120 with the use of a wide frequency band microphone (or, as shown, a hydrophone H, 250). The acoustic hydrophone 250 is, as an example, sensitive to frequencies that are lower than about 300 kHz. To generate the wave 240 at a chosen frequency fₘ, the ultrasound beam emanating from the RFT 210 can be modulated, in one implementation, with a sinusoidal waveform at such frequency. In a related implementation, to produce a transient URF, the RFT 210 can be utilized in a pulsed mode. The bone 120 operates as a main channel for transmission of acoustic wave 240 because the URF is generated mostly at a surface of the bone 240. Upon propagation through the bone 120, the wave 240 is influenced by viscoelastic properties of the bone 120. Accordingly, the wave 244 contains a wave signature representing portions of the bone 120 that are characterized by modified or altered mineralization and, for that reason, having different viscoelastic characteristics. Consequently, the analysis of spectral and temporal content of the registered wave 244 produces data representing spatial distribution of the bone structure and, in particular, mineralization of the bone 120.

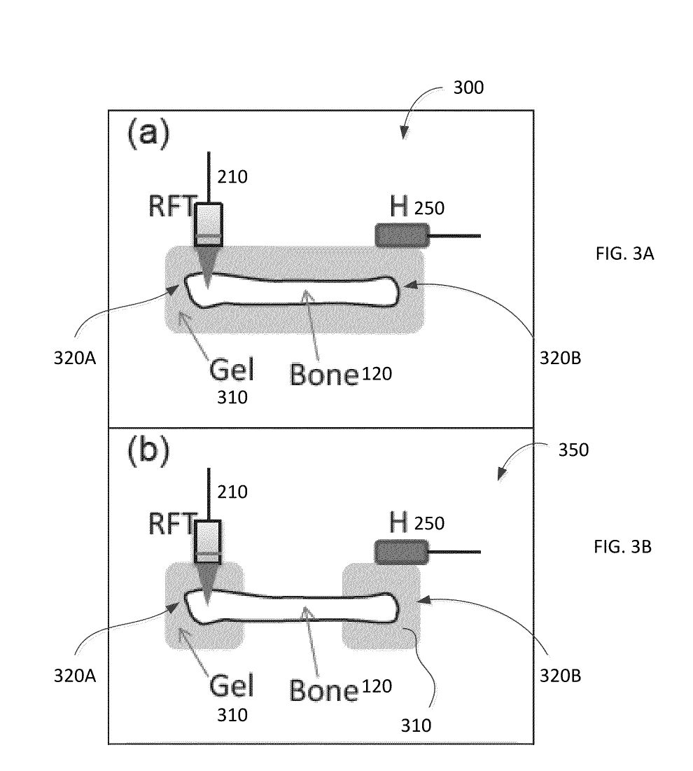

FIGS. 3A and 3B are diagrams representing schematically an experimental setup adapted for remote generation of acoustic waves, with the use of the RFT 210 in the bone 120 and the detection of the waves that propagated through the bone 120 with the hydrophone 250. In one embodiment, the RFT 210, operating within a band with a central frequency of about 3 MHz generated an impulse (˜2 microseconds long) radiation force at the bone 120, which radiation force cause the channeled acoustic wave 240 (shown in FIG. 2 but not in FIGS. 3A, 3B) to propagate through the bone 120 to be picked-up , at the other end of the bone, with the hydrophone 250 (Reson 4014) in a form of the registered, signature wave 244 (not shown). Both the RFT 210 and the hydrophone 250 were acoustically coupled to the bone 160 with tissue-mimicking gel 310 having acoustic characteristics that are similar to those of the tissue 160 surrounding the bone 120 (as shown in FIGS. 1, 2). The data representing the wave 244 detected by the hydrophone 250 have been processed with the use of a short-time Fourier transform to obtain a spectrogram of the wave 244. (Short time Fourier transform is obtained by successively taking Fourier transforms of overlapping short segments of a long time series. See, for example, Speech Enhancement in the STFT Domain (Springer Briefs in Electrical and Computer Engineering), by Jacob Benesty, Springer; 2012).

The data recordation was implemented in two cases: with the bone 120 substantially surrounded by and embedded in the gel 310, as shown in FIG. 3A, and with the gel 310 contacting the bone 120 only in the area of URF generation (input end 320A of the bone 120) and the area where the wave 244 was outcoupled from the bone 120 (output end 320B of the bone 120), as shown in FIG. 3B. In different experiments, rabbit femur bones with an approximately 10 cm length and a shaft of approximately oval cross-section of about 8-by-10 mm (in the middle of the bone) were tested in three states: a) intact; b) partially demineralized; and c) fully demineralized. Partial demineralization of the bone was approximated by soaking the bone in a 5% acetic acid solution for about 4 days. Full demineralization of the bone was approximated by soaking of the bone is such solution for about 7 days. The measurement of the intact bone was performed with the use of the embodiment 300 of FIG. 3A.

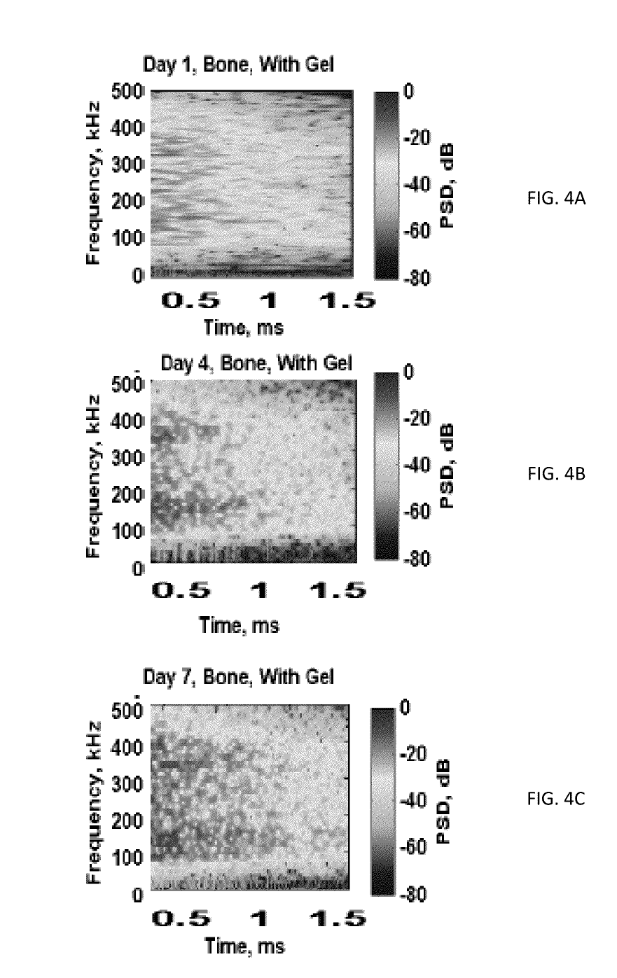

Spectrograms (expressed as color-maps in the coordinates of time and frequency) that correspond corresponding to the measurements performed with the use of the embodiment 300 of FIG. 3A, for each of the measurements a), b), and c), are shown in FIGS. 4A, 4B, and 4C respectively. In other words, the sequence of FIGS. 4A, 4B, and 4C provides illustration to changes in characteristics of acoustic wave propagation through the bone as a function of a degree of the bone's demineralization. A person of skill in the art will readily observe, from FIGS. 4A through 4C, that frequency range characterizing an acoustic wave detected at the output end of the bone is extended towards higher and higher frequencies as the degree of demineralization of the bone increases. Similarly, the spectrograms also extend further in time. It can be concluded, therefore, that a spectrogram representing an acoustic wave propagation through the bone can be used as an indicator of mineralization of the bone even in the presence of soft tissue (gel 310) around the bone.

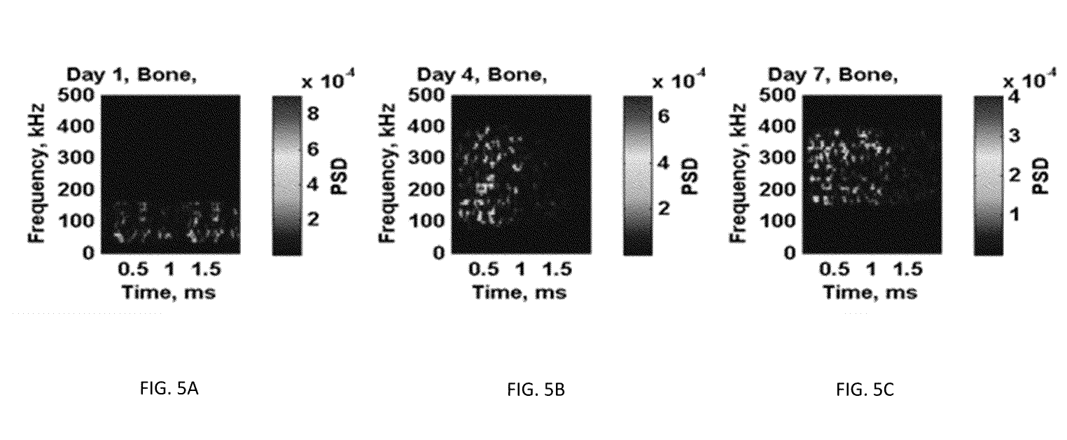

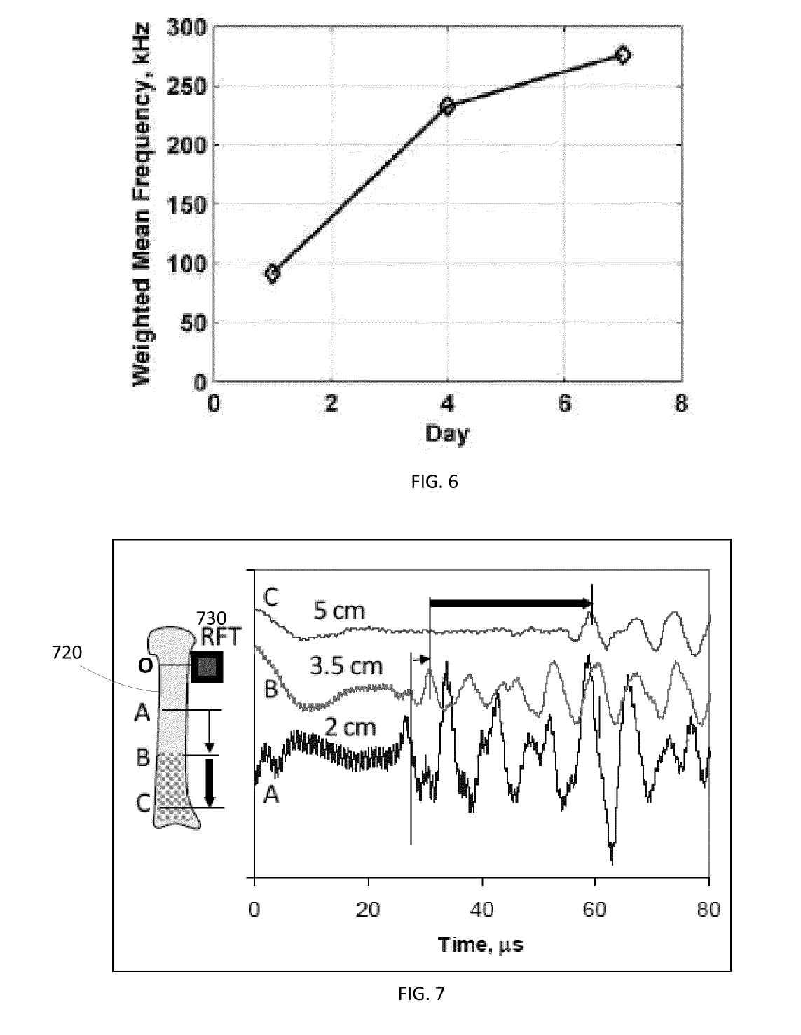

In further reference to FIGS. 3A and 3B, since the muscle and soft tissue surrounding the bone 120 from the input end 320A to the output end 320B (which in the experiment was mimicked by extending the gel 310 along the bone 120 from the input end 320A all the way to the output end 320B of the bone 120), a portion of the wave 244 may propagate towards the hydrophone along the soft tissue (gel 310). To separate the effect of acoustic wave propagation along the soft tissue surrounding the bone from the propagation of the wave through the bone itself, similar spectrograms of acoustic waves were measured, at the output of a bone with the use of the embodiment 350 of FIG. 3B, under the conditions corresponding to the degrees of bone-demineralization a), b), and c) as those of FIGS. 4A, 4B, 4C. The results are presented in FIGS. 5A, 5B, and 5C, respectively, and are consistent with those of FIGS. 4A through 4C in that the frequency content of the acoustic wave detected with the hydrophone 250 according to the embodiment 350 of FIG. 3B extends towards higher frequencies as demineralization of the bone increases. FIG. 6 presents a plot of dependency of a mean frequency of the spectrum of the wave 244 detected with the embodiment 350 as a function of time (which, in practice, as discussed above, represents a degree of bone demineralization, with time “day 1” corresponding to an intact bone). Empirical results indicate that the mean frequency shifted from about 90 kHz (for an intact bone) to about 275 kHz (for a fully demineralized bone). While the mean-frequency shift may vary from one measurement to another and one bone to another, the demonstrated more than three-fold increase of the mean spectral frequency of the acoustic wave that the bone transmits provides a clear demonstration that a change in a demineralization condition of a bone can be detected by analyzing a spectral pattern of an acoustic wave propagating through the bone. Based on such analysis, a user is enabled to discriminate between a mineralized bone and a demineralized bone.

In another implementation, the results of measurements in which are presented schematically in a diagram and plot of FIG. 7, the intact and decalcified chicken bones were used as an approximation to bones of newborns characterized by poor mineralization. (The size of chicken femur is comparable to the bones of infants.) The radiation force transducer RFP 730 was separated from the bone 720 by a layer of soft tissue and excited at about 1.6 MHz central frequency by an approximately 1 ms 100 V pulse. (The ultrasound safety of this experiment to the living tissue was confirmed by an estimate of the threshold of observable thermal coagulation effect associated with exposure of the bone to ultrasound, which indicated that the damaging effect may become apparent at exposures of about 1 second, or three orders of magnitude longer than the exposure time used.)

In preparation of a bone sample for the measurements with the embodiment of FIG. 7, the demineralization of a part of the bone (shown as a hatched portion of the bone 720 between the points B and C, which corresponds to about ⅓ of the total length of the bone 720) was achieved by submerging such part of the bone in the 25% EDTA solution for 4 days. The plots of FIG. 7 show the acoustic signals registered with the hydrophone 250 from the locations, at the bone, in the vicinity of points A, B, and C equidistantly-defined along the bone 720 at about 2, 3.5, and 5 cm from the location of the point O, where the operating RFT 730 applied the URF to the bone 720. The arrival time corresponding to each of the waves is indicated with a corresponding arrow. The time-of-flight (TOF) for the acoustic wave that has propagated through the decalcified zone (from point B to point C) is much longer that the TOF representing the acoustic wave propagation through the intact portion of the bone (from point A to point B). Accordingly, the empirical results of FIG. 7 are indicative of the feasibility to detect changes in bone mineralization condition by analyzing a temporal pattern characterizing the acoustic wave propagation through the bone.

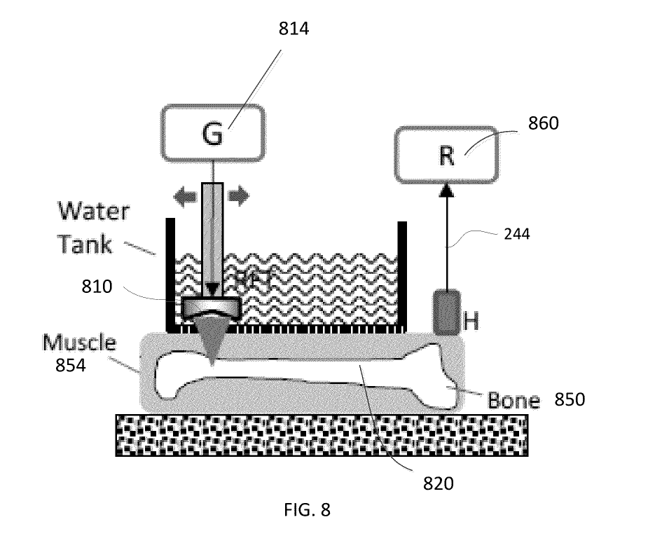

An alternative embodiment 800 of an ultrasound system configured for bone evaluation is shown in FIG. 8. Here, the radiation force transducer 810 is driven by a signal generator (G, 814) enabled to produce waveforms (including but not limited to a short tone burst and/or a waveform that is amplitude-modulated at a chosen frequency fₘ, for example within the range between about 50 kHz and about 500 kHz) to generate a radiation force at the bone 820. The applied to the bone 820 radiation force generally depends on a wavefront driving the RFT 810 and may include, as non-limiting examples, a wide spectral band impulse force and a harmonic radiation force characterized by the frequency fₘ. (Alternatively or in addition, the RFT 810 may be used in a pulse-echo mode for measuring a distance from the transducer 810 to the bone 820.) The RFT 810 is preferably disposed on a positioning stage (not shown) to allow for acoustic interrogation across the surface of the bone and, in particular, along the bone length. Optionally, the acoustic wave transmitted from the RFT 810 towards the bone 820 is focused onto to the bone surface (for example, by appropriately configuring the transducer array of the RFT 810 in space or by introducing appropriate phase delays between and among the waves transmitted by individual elements of the RFT 810. In addition, the acoustic beam generated by the RFT 810 can be directed to a pre-determined position along the bone structure 820, as discussed below). The hydrophone 850 is positioned in acoustic communication with the bone 820 and/or the muscle 854 to receive, as a function of position along the bone, an acoustic wave transmitted along the bone 820 as a result of each occurrence of URF excitation of the bone by the RFT 810. The signal acquired with the hydrophone 850 is passed on to the receiver R, 860. The processor of the system (not shown), which may be programmed to control at least one of the RFT 810 and the hydrophone 850, is further used to digitize and record the data acquired with the hydrophone 850 for further processing.

In a related implementation of the system, the signal generator is configured and/or programmed to drive the source of ultrasound in one of a short-pulse mode (corresponding to generation of a few cycles of ultrasound long that act as an impulse of energy applied to the object to induce a wide range of vibration frequencies in the object), a tone-burst mode (many cycles of ultrasound long that, optionally, can be of constant amplitude, to deposit more energy in the object than just a short pulse), and an amplitude-modulated tone-burst mode (many cycles of ultrasound with amplitude modulation, where modulation can be in various forms including a sinusoidal form to induce vibration, in the object, at one or more specific low frequency(ies).

As a result of acoustic data processing and according to an algorithm discussed, for example, by Steven Kay (in Modern Spectral Estimation: Theory and Application, Prentice Hall), the acoustic wave spectrograms are formed to characterize the frequency composition of the acoustic wave that has propagated through the bone structure and to estimate a mean frequency shift associated with bone demineralization condition that is changing on a long time scale. In addition or alternatively, an acoustic wave arrival time (time of flight) as a function of position along the bone structure is measured to analyze transient characteristics of the bone structure and assesses the geometry of bone demineralization.

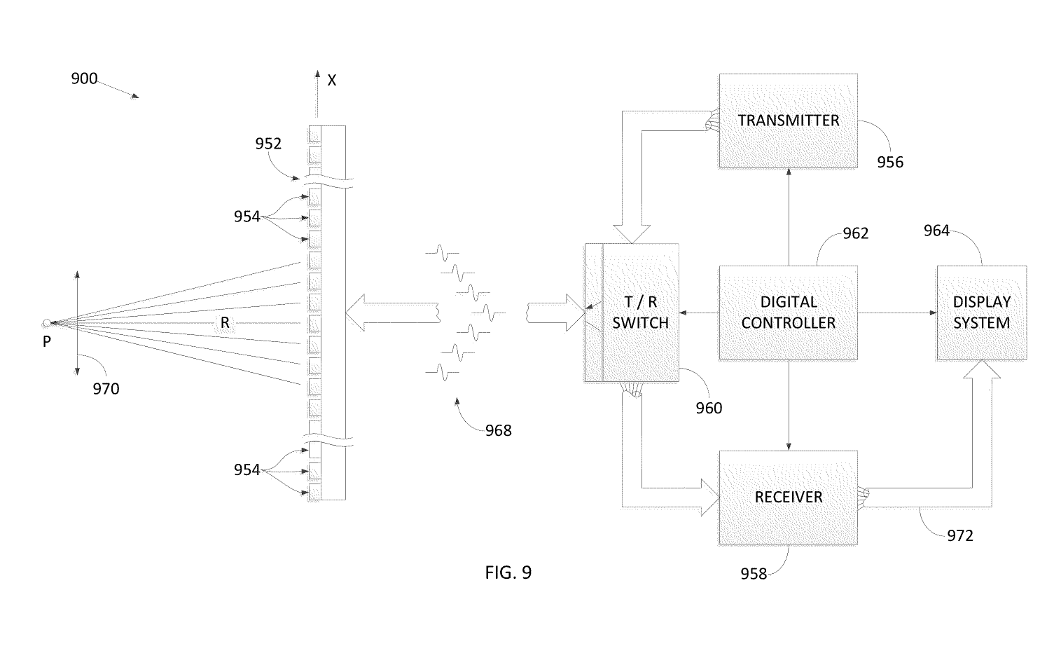

An example of an ultrasound subsystem 900 that, in one embodiment, is used to form at least a part of the RFT 810 is shown in FIG. 9. The subsystem 900 includes a transducer array 952 containing a plurality of separately driven elements 954 each of which produces a burst of ultrasonic energy when energized by a pulse produced by a transmitter 956. (In some embodiments, the ultrasonic energy that is reflected back to the transducer array 952 from the tissue of interest may be converted to an electrical signal by each transducer element 954 and applied separately to a receiver 958 through a set of switches 960. The transmitter 956, and the receiver 958 and the switches 960, when present, are operated under the control of a digital controller 962 responsive to the commands input by the human operator. In this case, a complete scan can be performed by acquiring a series of echoes in which the switches 960 are set to their transmit position, the transmitter 956 is gated on momentarily to energize each transducer element 954, the switches 960 are then set to their receive position, and the subsequent echo signals produced by each transducer element 954 are applied to the receiver 958. The separate echo signals from each transducer element 954 are combined in the receiver 958 to produce a single echo signal which is employed to produce a line in an image on a display system 964.)

The transmitter 956 drives the transducer array 952 such that an ultrasonic beam is produced which is directed substantially perpendicular to its front surface. To focus this beam at a range, R, from the transducer 952 a subgroup of the elements 954 are energized to produce the beam, and the pulsing of the inner elements in this subgroup 954 are delayed relative to the outer elements of 954 as shown at 968. A beam focused at point P results from the interference of the small separate wavelets produced by the subgroup elements. The time delays determine the depth of focus, or range R, and this is typically changed if a scan of the beam across the space is required. In such case, the subgroup of elements to be energized can be shifted one element position along the transducer length. As indicated by the arrow 970, the focal point, P, of the ultrasonic beam is thus shifted along the length of the transducer 952 by repeatedly shifting the location of the energized subgroup of elements 954. The term “focal point,” as referred to herein, includes not only a single point object in the usual sense, but also a general region-of-interest to which ultrasound energy is delivered in a substantially focused manner.

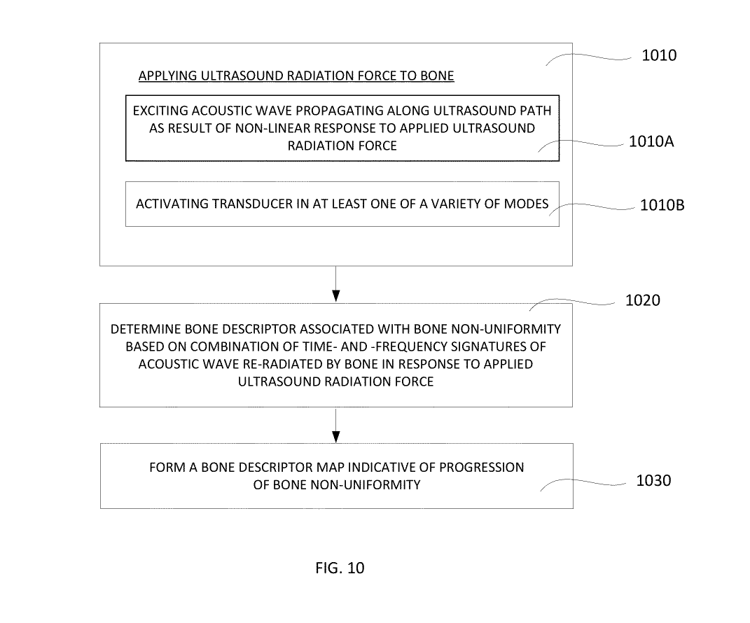

FIG. 10 illustrates schematically an embodiment of a method for assessment of a bone non-uniformity according to the invention. Here, at step 1010 bone is exposed to an ultrasound radiation force, which excites an acoustic wave to propagate along the bone at step 1010A. The application of the ultrasound radiation force can be effectuated by activating a transducer externally to the bone, at step 1010C, in a variety of modes. The bone descriptor associated with bone non-uniformity is determined at step 1020 and, optionally, a map of such bone descriptor is created at step 103 by measuring the bone descriptor as a function of at least one of time and space (along the bone) to determine a progression of bone non-uniformity in time and/or along the bone. The non-uniformity may be indicative of a bone disease. In one specific implementation, a method includes (i) externally applying an ultrasound radiation force to the bone and (ii) with the use of an acoustic detector (such as a hydrophone) determining a bone-structure descriptor associated with the bone non-uniformity based on a combination of a time-signature and a wide-spectral-band frequency signature of an acoustic wave that has been radiated by the bone towards the acoustic detector as a result of a non-linear response of the bone to the applied ultrasound radiation force. The external application of the URF may include exciting, in the bone, an acoustic wave propagating along an ultrasound path along the bone. Alternatively or in addition, the external application of the URF may include activating a transducer to produce an acoustic pulse in one of a short pulse mode, a tone-burst mode, and an amplitude-modulated tine-burst mode. Alternatively or in addition, the external application of the URF to the bone may include an external application of the URF to the bone at multiple points along the bone, in which case the method further includes a step of generating a map of spatial distribution of the bone-structure descriptor in the bone. In addition, the method may include a determination of a time-dependency of the bone-structure descriptor (by determining such descriptor at different points in time) to generate an output indicative of a time-progression of the bone non-uniformity. A process of determining a bone-structure descriptor according to an embodiment of the invention may be devoid of determining a speed-of-sound in the bone.

References throughout this specification to “one embodiment,” “an embodiment,” “a related embodiment,” or similar language mean that a particular feature, structure, or characteristic described in connection with the referred to “embodiment” is included in at least one embodiment of the present invention. Thus, appearances of the phrases “in one embodiment,” “in an embodiment,” and similar language throughout this specification may, but do not necessarily, all refer to the same embodiment. It is to be understood that no portion of disclosure, taken on its own and in possible connection with a figure, is intended to provide a complete description of all features of the invention.

In addition, the following disclosure may describe features of the invention with reference to corresponding drawings, in which like numbers represent the same or similar elements wherever possible. In the drawings, the depicted structural elements are generally not to scale, and certain components may be enlarged and not necessary properly oriented relative to the other components for purposes of emphasis and understanding. It is to be understood that no single drawing is intended to support a complete description of all features of the invention. In other words, a given drawing is generally descriptive of only some, and generally not all, features of the invention. A given drawing and an associated portion of the disclosure containing a description referencing such drawing do not, generally, contain all elements of a particular view or all features that can be presented is this view, for purposes of simplifying the given drawing and discussion, and to direct the discussion to particular elements that are featured in this drawing. A skilled artisan will recognize that the invention may possibly be practiced without one or more of the specific features, elements, components, structures, details, or characteristics, or with the use of other methods, components, materials, and so forth. Therefore, although a particular detail of an embodiment of the invention may not be necessarily shown in each and every drawing describing such embodiment, the presence of this detail in the drawing may be implied unless the context of the description requires otherwise. In other instances, well known structures, details, materials, or operations may be not shown in a given drawing or described in detail to avoid obscuring aspects of an embodiment of the invention that are being discussed. Furthermore, the described single features, structures, or characteristics of the invention may be combined in any suitable manner in one or more further embodiments.

If the schematic flow chart diagram is included, it is generally set forth as a logical flow-chart diagram. As such, the depicted order and labeled steps of the logical flow are indicative of one embodiment of the presented method. Other steps and methods may be conceived that are equivalent in function, logic, or effect to one or more steps, or portions thereof, of the illustrated method. Additionally, the format and symbols employed are provided to explain the logical steps of the method and are understood not to limit the scope of the method. Although various arrow types and line types may be employed in the flow-chart diagrams, they are understood not to limit the scope of the corresponding method. Indeed, some arrows or other connectors may be used to indicate only the logical flow of the method. For instance, an arrow may indicate a waiting or monitoring period of unspecified duration between enumerated steps of the depicted method. Without loss of generality, the order in which processing steps or particular methods occur may or may not strictly adhere to the order of the corresponding steps shown.

The invention as recited in claims appended to this disclosure is intended to be assessed in light of the disclosure as a whole.

The present invention has been described in terms of one or more preferred embodiments, and it should be appreciated that many equivalents, alternatives, variations, and modifications, aside from those expressly stated, are possible and within the scope of the invention. For example, the processing of the recorded data that represents bone density is alternatively or in addition accomplished with the use of the signal analysis algorithm transforms the data obtained from a spectrogram of the signal with the use of short-time Fourier Transform to extract physical parameters describing bone density. Alternatively or in addition, the signal processing algorithm is configured to analyze relative signal intensity in time-frequency domain by comparing the amplitudes of the recorded spectrogram at different frequencies and at different times. The term “time-frequency” refers to a spectrogram that is a function of time and frequency. In particular, such algorithm is adapted to extract, from the recorded data, the rate at which signal at each frequency component decays as a function of time. In a related embodiment, the algorithm is adapted to produce the frequency-dependent decay characteristic of the amplitude of the signal at any given time. Mean arrival time can be calculated for the entire wave, or for each frequency component using the spectrogram. Time duration for each frequency component can be calculated as the length of time that each frequency component lasts. In a related embodiment, the used data processing algorithm operates with the use of wavelet coefficients obtained by the wavelet transform (see, for example, Ram Shankar Pathak, The Wavelet Transform (Atlantis Studies in Mathematics for Engineering and Science, Atlantic Press, 2009.

1. An ultrasound system for assessment of a structural parameter of a bone of a subject, the system comprising:
a signal generator;
a source of ultrasound wave, said source being driven by the signal generator and oriented repositionably and distantly with respect to the subject to apply an ultrasound radiation force to an identified input location at the bone to create an acoustic wave propagating along the bone;
an acoustic detector positioned moveably along the bone to detect said acoustic wave at a first and second output positions along the bone; and
a data-processing circuitry receiving an output from the acoustic detector and operable to record spectral and temporal data representing said acoustic waves based on such output and to process said spectral and temporal data to determine a structural parameter of the bone.

2. A system according to claim 1, wherein said structural parameter represents a change in a mineralization characteristic of said bone.

3. A system according to claim 1, wherein said source is configured to transmit a substantially focused ultrasound beam to the bone.

4. A system according of claim 1, wherein the acoustic detector is structured to be placed in contact with skin of the subject.

5. A system according to claim 1, wherein the signal generator includes electronic circuitry structured to drive the source of ultrasound in one of a short pulse mode; a tone-burst mode, and an amplitude-modulated tone-burst mode.

6. A system according to claim 1, wherein said spectral and temporal data include at least one of a mean change in frequency as a function of time, a parameter representing a broadening of a frequency spectrum as a function of time, a mean time of arrival of said acoustic wave from the identified input location to a position along the bone at which said acoustic wave is detected, a parameter representing a change in a temporal characteristic of said acoustic wave as a function of frequency, and a change in the amplitude of said acoustic wave determined in a frequency domain.

7. A method for assessment of a bone non-uniformity, the method comprising:
externally applying an ultrasound radiation force to the bone; and
with the use of an acoustic detector, determining a bone-structure descriptor associated with the bone non-uniformity based on a combination of a time-signature and a wide-spectral-band frequency signature of an acoustic wave that has been radiated by the bone towards the acoustic detector as a result of a non-linear response to the applied ultrasound radiation force.

8. A method according to claim 7, wherein the externally applying includes exciting, in the bone, a first acoustic wave propagating along an ultrasound path along the bone.

9. A method according to claim 7, wherein the externally applying includes activating a transducer to produce an acoustic pulse in one of a short pulse mode; a tone-burst mode, and an amplitude-modulated tone-burst mode.

10. A method according to claim 7, wherein the externally applying includes externally applying an ultrasound radiation force to the bone at multiple points along the bone, and
further comprising:
generating a map of spatial distribution of the bone-structure descriptor in the bone.

11. A method according to claim 7, wherein the determining includes determining of a time-dependent bone-structure descriptor at first and second time to generate an output indicative of progression of the bone non-uniformity between the first and second time.

12. A method according to claim 7, wherein the bone non-uniformity is indicative of a bone disease.

13. A method according to claim 7, wherein the determining the bone structure descriptor is devoid of determining a speed-of-sound in the bone.
